SEQUENCE LISTING (18 sequences)

SEQ ID NO: 1 - DNA, 64 bp, Artificial sequence
  Synthetic oligonucleotide
gatcccctgtcaactcccccgacagcttcaagagagctgtcgggggagttgacatttttggaaa

SEQ ID NO: 2 - DNA, 64 bp, Artificial sequence
  Synthetic oligonucleotide
agcttttccaaaaatgtcaactcccccgacagctctcttgaagctgtcgggggagttgacaggg

SEQ ID NO: 3 - DNA, 22 bp, Artificial sequence
  Synthetic primer
caggcggtgaaggagatgaacg

SEQ ID NO: 4 - DNA, 25 bp, Artificial sequence
  Synthetic reverse Primer
aaatccacgatagagtagttggcgc

SEQ ID NO: 5 - DNA, 20 bp, Artificial sequence
  Synthetic probe
cccgagccagcccaacgagg

SEQ ID NO: 6 - protein, 20 aa, Homo sapiens
  Misc_feature (4)..(4): Xaa can be any naturally occurring amino acid
DKNXGQEGYIPSNYVTEAED

SEQ ID NO: 7 - protein, 20 aa, Homo sapiens
  Misc_feature (3)..(4): Xaa can be any naturally occurring amino acid
VNXXDRQGFVPAAYVKKLDX

SEQ ID NO: 8 - protein, 20 aa, Homo sapiens
  Misc_feature (19)..(20): Xaa can be any naturally occurring amino acid
SLTTGQTGYIPSNYVAPSXX

SEQ ID NO: 9 - protein, 20 aa, Homo sapiens
SLTTGETGYIPSNYVAPVDS

SEQ ID NO: 10 - protein, 20 aa, Homo sapiens
  Misc_feature (4)..(4): Xaa can be any naturally occurring amino acid
TKNXGXQGWVPSNYITPVSX

SEQ ID NO: 11 - protein, 20 aa, Homo sapiens
  Misc_feature (3)..(4): Xaa can be any naturally occurring amino acid
LNXXGKDGFIPKNYIEMKPH

SEQ ID NO: 12 - protein, 20 aa, Homo sapiens
  Misc_feature (3)..(4): Xaa can be any naturally occurring amino acid
CHXXGQTGMFPRNYVTPVNR

SEQ ID NO: 13 - protein, 20 aa, Homo sapiens
  Misc_feature (3)..(4): Xaa can be any naturally occurring amino acid
LGXXDREGYVPKNLLGLYPR

SEQ ID NO: 14 - protein, 20 aa, Homo sapiens
  Misc_feature (3)..(4): Xaa can be any naturally occurring amino acid
LNXXDKEGYVPRNLLGLYPR

SEQ ID NO: 15 - protein, 20 aa, Homo sapiens
  Misc_feature (3)..(4): Xaa can be any naturally occurring amino acid
LHXXGQEGYVPRNYFGLFPR

SEQ ID NO: 16 - protein, 350 aa, Homo sapiens
MWMKDPVARPLSPTRLQPALPPEAQSVPELEEVARVLAEIPRPLKRRGSMEQAPAVALPPTHKKQYQQIISRLFHRHGGPGPGGRSQSCPPSLRDLRPGQGPLLLPHQLPFHRPAPSQSSPPEQPQSMEMRSVLRKAGSPRKARRARLNPLVLLLDAALTGELEVVQQAVKEMNDPSQPNEEGITALHNAICGANYSIVDFLITAGANVNSPDSHGWTPLHCAASCNDTVICMALVQHGAAIFATTLSDGATAFEKCDPYREGYADCATYLADVEQSMGLMNSGAVYALWDYSAEFGDELSFREGESVTVLRRDGPEETDWWWAALHGQEGYVPRNYFGLFPRVKPQRSK

SEQ ID NO: 17 - DNA, 2156 bp, Homo sapiens
gcggccgcgtcgacccggcgttcagacgcgggcagctaccggcgctcgctgggctccgcggggccgtcgggcactttgcctcgcagctggcagcccgtcagccgcatccccatgcccccctccagcccccagccccgcggggccccgcgccagcgtcccatccccctcagcatgatcttcaagctgcagaacgccttctgggagcacggggccagccgcgccatgctccctgggtcccccctcttcacccgagcacccccgcctaagctgcagccccaaccacaaccacagccccagccacaatcacaaccacagccccagctgccccaacagccccagacccaaccccaaacccctaccccagcctcccacatccgcatccccaacagacatggccccctgtgaacgaaggaccccccaaaccccccaccgagctggagcctgagccggagatagaggggctgctgacaccagtgctggaggctggcgatgtggatgaaggaccctgtagcaaggcctctcagccccacgaggctgcagccagcactgccaccggaggcacagtcggtgcccgagctggaggaggtggcacgggtgttggcggaaattccccggcccctcaaacgcaggggctccatggagcaggcccctgctgtggccctgccccctacccacaagaaacagtaccagcagatcatcagccgcctcttccatcgtcatggggggccagggcccggggggcggagccagagctgtcccccatcactgagggatctgaggccagggcagggccccctgctcctgccccaccagctcccattccaccgcccggccccgtcccagagcagcccaccagagcagccgcagagcatggagatgcgctctgtgctgcggaaggcgggctccccgcgcaaggcccgccgcgcgcgcctcaaccctctggtgctcctcctggacgcggcgctgaccggggagctggaggtggtgcagcaggcggtgaaggagatgaacgacccgagccagcccaacgaggagggcatcactgccttgcacaacgccatctgcggcgccaactactctatcgtggatttcctcatcaccgcgggtgccaatgtcaactcccccgacagccacggctggacacccttgcactgcgcggcgtcgtgcaacgacacagtcatctgcatggcgctggtgcagcacggcgctgcaatcttcgccaccacgctcagcgacggcgccaccgccttcgagaagtgcgacccttaccgcgagggttatgctgactgcgccacctacctggcagacgtcgagcagagtatggggctgatgaacagcggggcagtgtacgctctctgggactacagcgccgagttcggggacgagctgtccttccgcgagggcgagtcggtcaccgtgctgcggagggacgggccggaggagaccgactggtggtgggccgcgctgcacggccaggagggctacgtgccgcggaactacttcgggctgttccccagggtgaagcctcaaaggagtaaagtctagcaggatagaaggaggtttctgaggctgacagaaacaagcattcctgccttccctccagacctctccctctgttttttgctgcctttatctgcacccctcaccctgctggtggtggtccttgccaccggttctctgttctcctggaagtccagggaagaaggagggccccagccttaaatttagtaatctgccttagccttgggaggtctgggaagggctggaaatcactggggacaggaaaccacttccttttgccaaatcagatcccgtccaaagtgcctcccatgcctaccaccatcatcacatcccccagcaagccagccacctgcccagccgggcctgggatgggccaccacaccactggatattcctgggagtcactgctgacaccatctctcccagcagtcttggggtctgggtgggaaacattggtctctaccaggatccctgccccacctctccccaattaagtgccttcacacagcactggtttaatgtttataaacaaaatagagaaactggtttaatgtttataaacaaaatagagaaactttcgcttataaataaaagtagtttgcacagaaatgaaaaaaaaaaaaaaaaaaaaaaaa

SEQ ID NO: 18 - protein, 393 aa, Homo sapiens [sequence omitted]

(19) United States
(12) Patent Application Publication
(10) Pub. No.: US 2008/0193488 A1
(43) Pub. Date: Aug. 14, 2008

(54) Variant Polypeptide and Screening Assay

(75) Inventor: Xin Lu, London (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Ludwig Institute for Cancer Research, New York, NY (US)

(21) Appl. No.: 11/995,669

(22) PCT Filed: Jul. 13, 2006
(86) PCT No.: PCT/EP2006/006850

§ 371 Date: Feb. 20, 2008

(30) Foreign Application Priority Data
Jul. 14, 2005 (GB) 0514538.8

(51) Int. Cl. A61K 39/00; C12N 15/11; C12N 15/00; C07K 14/00; C07K 16/18; A61P 37/00; A61K 38/00; A61K 31/70; G01N 33/00; G01N 33/566

(52) U.S. Cl. 424/277.1; 536/23.1; 435/320.1; 530/350; 514/44; 436/86; 436/501; 424/184.1; 530/387.7; 514/12

(57) ABSTRACT

The invention includes a screening assay for the identification of agents that inhibit the interaction of p53 with p53 inhibitory polypeptides, and p53 inhibitory polypeptide variants that are inactive with respect to the inhibition of p53 activity.

The invention relates to a screening assay for the identification of agents that inhibit the interaction of p53 with p53 inhibitory polypeptides and including p53 inhibitory polypeptide variants that are inactive with respect to the inhibition of p53 activity.

Tumour suppressor genes encode proteins which function to inhibit cell growth or division and are therefore crucially important with respect to maintaining proliferation, growth and differentiation of normal cells. Mutations in tumour suppressor genes result in abnormal cell-cycle progression whereby the normal cell-cycle check points which arrest the cell-cycle, when, for example, DNA is damaged, are ignored and damaged cells divide uncontrollably. Arguably, the tumour suppressor gene which has been the subject of the most intense research is p53.

The p53 gene encodes a protein which functions as a transcription factor and is a key regulator of the cell division cycle. It was discovered as a protein shown to bind with affinity to the SV40 large T antigen. The p53 gene encodes a 393 amino acid polypeptide with a molecular weight of 53 kDa. The identification of the ASPP family of proteins as specific regulators of p53 revealed a novel mechanism by which the apoptotic function of p53 is regulated¹ (see WO02/12325).

Whereas the apoptotic function of p53 is stimulated by ASPP1 and ASPP2², the related iASPP inhibits p53-dependent apoptosis 3. Regulation of p53 by the ASPP family members is evolutionarily conserved from worm to human^{2 3} and is disclosed in currently unpublished PCT application PCT/GB04/003492 which is incorporated by reference. Moreover, the involvement of the ASPP family in tumour development is reflected by the observation that the expression of ASPP1 and ASPP2 is often reduced while iASPP is increased in a large percentage of tumours examined^{2 3-5}. An inverse relationship between ASPP2 expression and clinical outcome of B-cell lymphomas was also observed⁶.

The mechanistic basis by which ASPP1 and ASPP2 are activators of p53 while iASPP is an inhibitor of p53 remains unclear.

All ASPP family members bind p53 through their C-terminus. Furthermore, the most homologous region among the ASPP family members is located within its C-terminus and it carries the signature sequences of this family of proteins; Ankryin repeats, SH3 domain and Proline rich region containing Protein (ASPP). ASPP1 and ASPP2 belong to a unique class of SH3 domain containing proteins in which one of the critical proline contact residues in the SH3 domain of ASPP1 and ASPP2 is changed from Tyr to Leu. Based on the analysis of the co-crystal structure of the DNA binding domain of p53 and the SH3 domain of ASPP2 it was shown that this change allows ASPP2 to have higher binding affinity to the DNA binding domain of p53.

Moreover, p53 contains a proline rich sequence with five PXXP motifs which is known to be required for p53 to induce apoptosis but not cell cycle arrest⁷⁻⁹. The proline rich region of p53 is required for p53-dependent transactivation of target genes such as PIG3 but not p21waf1 or mdm2^{7,10}. Similarly, ASPP1 and ASPP2 selectively enhance the ability of p53 to transactivate pro-apoptotic genes such as Bax and PIG3 but not p21waf1 or mdm2².

Additionally, the most common polymorphism of p53 specifically found in human is located within the proline rich region of p53 at codon 72. In humans, the naturally occurring amino acid is either Proline (Pro) or Arginine (Arg). However the majority of vertebrates has Proline in the corresponding residue¹¹. Extensive studies have been carried out in the last decade to investigate the link between the expression of p53 polymorphic variants at codon 72 (p53Pro72 and p53Arg72) and cancer susceptibility. However, the outcome has been disappointing because of a lack of understanding of how p53Pro72 and p53Arg72 function in vivo¹²⁻¹⁷. Interestingly, the proline at residue 72 of p53 is part of a PXXP motif which is known to be critical in contacting the Tyr residue of the SH3 domain containing protein.

In our co-pending application PCT/GB04/004341, currently unpublished, which is incorporated by reference, we disclose, amongst other things, methods to screen for agents that modulate the interaction of iASPP with p53 and the preferential binding of iASPP and ASPP1/2 for the p53 polymorphic variant p53Pro72. A conserved Tyr and Leu in the SH3 domain of iASPP and ASPP2 determines their distinct binding preference to the proline-rich region and DNA binding domain of p53 respectively. The proline-rich region of p53 is required for the ASPP family members to regulate p53-mediated apoptosis. Importantly, the ASPP family members, particularly iASPP, bind and regulate the activities of p53Pro72 more efficiently than that of p53Arg72. Endogenous iASPP level dictates the activities of codon 72 polymorphic p53 and over-expression of iASPP occurs more frequently in tumours homozygous for p53Pro72 than for p53Arg72. Hence escape from the negative regulation of iASPP is one of the mechanisms by which p53Arg72 activates apoptosis more efficiently than p53Pro72.

We describe a further screening method that allows the identification of agents that inhibit the interaction of iASPP polypeptides that comprise the SH3 domain of iASPP with the proline rich domain of p53. We also describe variant iASPP polypeptides which show reduced binding to p53, in particular p53Pro72.

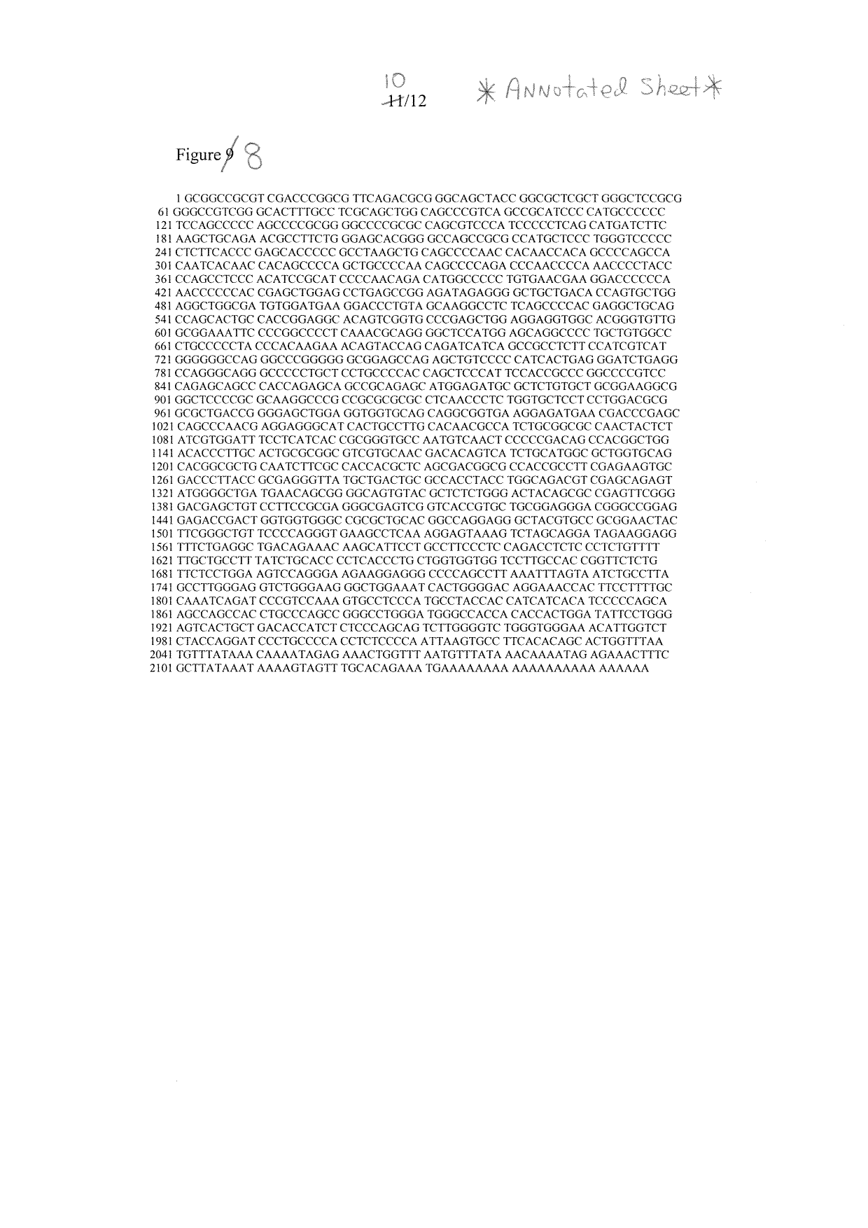

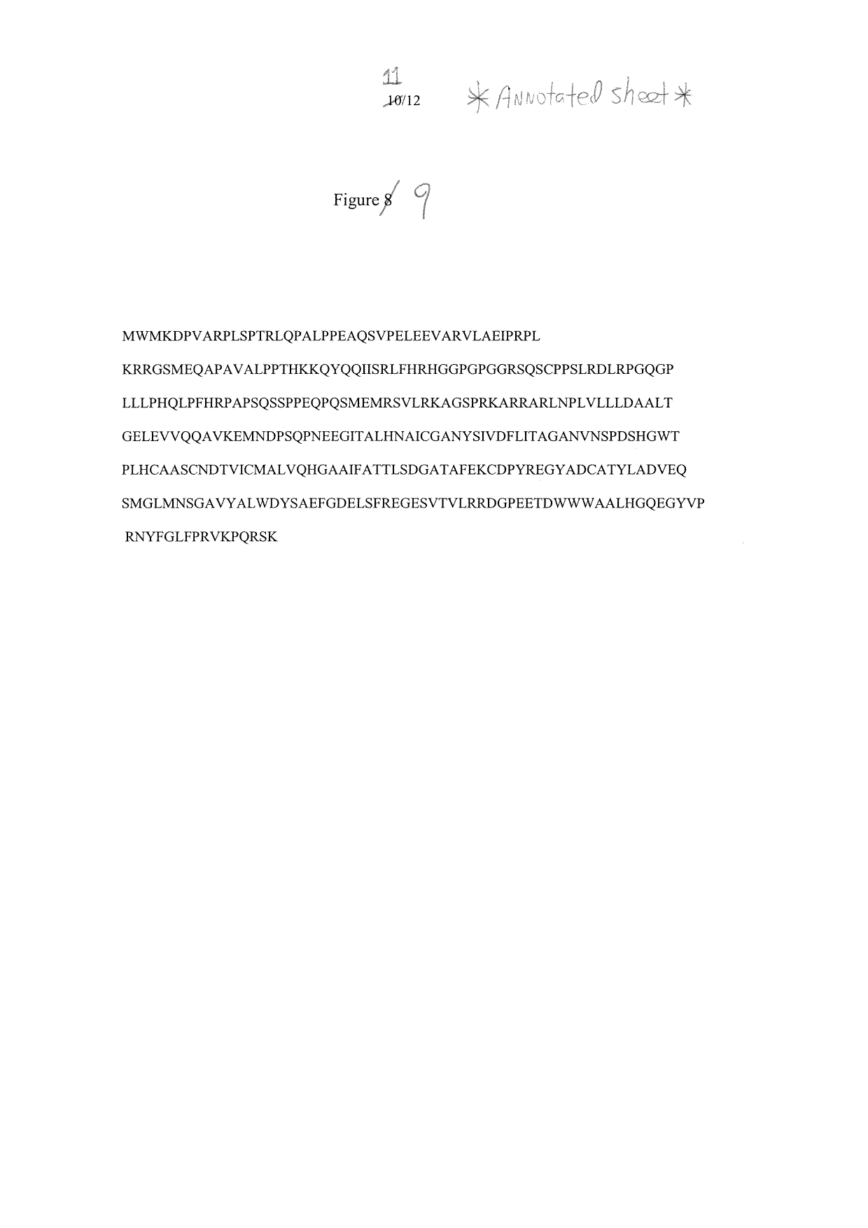

According to an aspect of the invention there is provided an isolated nucleic acid molecule as represented by the nucleic acid sequence in FIG. 8, or a nucleic acid molecule that hybridises under stringent hybridisation conditions to a nucleic acid molecule as represented in FIG. 8, wherein said nucleic acid is modified at a nucleotide codon that encodes for a tyrosine amino acid residue at position 814 as represented by the amino acid sequence represented in FIG. 9.

In a preferred embodiment of the invention said nucleic acid molecule comprises the nucleic acid sequence represented in FIG. 8. Preferably said nucleic acid molecule consists of the nucleic acid sequence as represented in FIG. 8.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecules are part of an expression vector, preferably an expression vector adapted for eukaryotic gene expression.

Typically said adaptation includes, by example and not by way of limitation, the provision of transcription control sequences (promoter sequences) which mediate cell/tissue specific expression. These promoter sequences may be cell/tissue specific, inducible or constitutive.

“Promoter” is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of physiological/environmental cues which include, by example and not by way of limitation, intermediary metabolites (eg glucose, lipids), environmental effectors (eg light, heat,).

Promoter elements also include so called TATA box and RNA polymerase initiation selection sequences which function to select a site of transcription initiation. These sequences also bind polypeptides which function, inter alia, to facilitate transcription initiation selection by RNA polymerase.

Adaptations also include the provision of selectable markers and autonomous replication sequences which facilitate the maintenance of said vector in either the eukaryotic cell or prokaryotic host. Vectors which are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) which function to maximise expression of vector encoded genes arranged in bi-cistronic or multi-cistronic expression cassettes. Expression control sequences also include so-called Locus Control Regions (LCRs). These are regulatory elements which confer position-independent, copy number-dependent expression to linked genes when assayed as transgenic constructs. LCRs include regulatory elements that insulate transgenes from the silencing effects of adjacent heterochromatin, Grosveld et al., Cell (1987), 51: 975-985.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al. U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al. (1997) J. Virol 71(9):7128-7131; Lee, et al. (1997) Mol. Cell. Biol. 17(9):5097-5105; and Dreher, et al. (1997) J. Biol. Chem 272(46); 29364-29371.

The viruses may also be designed to be selectively replicating viruses. Particularly preferred selectively replicating viruses are described in WO00/22137 and WO00/22136.

It has been demonstrated that viruses which are attenuated for replication are also useful in gene therapy. For example the adenovirus dl1520 containing a specific deletion in the E1b55K gene (Barker and Berk (1987) Virology 156: 107) has been used with therapeutic effect in human beings. Such vectors are also described in McCormick (U.S. Pat. No. 5,677,178 issued Oct. 14, 1997) and McCormick, U.S. Pat. No. 5,846,945 issued Dec. 8, 1998. The present invention may also be used in combination with the administration of such vectors to minimize the pre-existing or induced humoral immune response to such vectors.

It may be valuable in some instances to utilize or design vectors to achieve introduction of the exogenous transgene in a particular cell type. Certain vectors exhibit a natural tropism for certain tissue types. For example, vectors derived from the genus herpesviridiae have been shown to have preferential infection of neuronal cells. Examples of recombinantly modified herpesviridiae vectors are disclosed in U.S. Pat. No. 5,328,688 issued Jul. 12, 1994. Cell type specificity or cell type targeting may also be achieved in vectors derived from viruses having characteristically broad infectivity's by the modification of the viral envelope proteins. For example, cell targeting has been achieved with adenovirus vectors by selective modification of the viral genome knob and fibre coding sequences to achieve expression of modified knob and fibre domains having specific interaction with unique cell surface receptors. Examples of such modifications are described in Wickham, et al (1997) J. Virol 71(11):8221-8229 (incorporation of RGD peptides into adenoviral fiber proteins); Amberg, et al. (1997) Virology 227:239-244 (modification of adenoviral fiber genes to achieve tropism to the eye and genital tract); Harris and Lemoine (1996) TIG 12(10):400-405; Stevenson, et al. (1997) J. Virol. 71(6):4782-4790; Michael, et al. (1995) Gene Therapy 2:660-668 (incorporation of gastrin releasing peptide fragment into adenovirus fiber protein); and Ohno, et al. (1997) Nature Biotechnology 15:763-767 (incorporation of Protein A-IgG binding domain into Sindbis virus). Other methods of cell specific targeting have been achieved by the conjugation of antibodies or antibody fragments to the envelope proteins (see, e.g. Michael, et al. (1993) J. Biol. Chem 268:6866-6869, Watkins, et al. (1997) Gene Therapy 4:1004-1012; Douglas, et al. (1996) Nature Biotechnology 14: 1574-1578. Alternatively, particularly moieties may be conjugated to the viral surface to achieve targeting (See, e.g. Nilson, et al. (1996) Gene Therapy 3:280-286 (conjugation of EGF to retroviral proteins)). Additionally, the virally encoded therapeutic transgene also be under control of a tissue specific promoter region allowing expression of the transgene preferentially in particular cell types.

According to a further aspect of the invention there is provided an isolated variant polypeptide comprising an amino acid sequence wherein said polypeptide is modified by the deletion or substitution of at least amino acid residue tyrosine 814 of the amino acid sequence represented in FIG. 8.

A variant polypeptide may differ in amino acid sequence by one or more substitutions, additions, deletions, truncations which may be present in any combination. Among preferred modifications are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

In addition, the invention features variant polypeptide sequences having at least 75% identity with the polypeptide sequence as hereindisclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequence illustrated herein.

In a preferred embodiment of the invention said tyrosine amino acid residue is substituted with a leucine amino acid residue.

Preferably said variant polypeptide is modified only at amino acid residue tyrosine 814. Preferably said variant polypeptide is a tyrosine for a leucine substitution.

According to a further aspect of the invention there is provided the use of a modified nucleic acid or modified polypeptide according the invention as a pharmaceutical.

According to a further aspect of the invention there is provide a pharmaceutical composition comprising a modified nucleic acid or modified polypeptide according to the invention.

When administered, the nucleic acids/polypeptides of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The nucleic acids/polypeptides the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of nucleic acid/polypeptided for producing the desired response in a unit of weight or volume suitable for administration to a patient. The response can, for example, be measured by determining regression of a tumour, decrease of disease symptoms, modulation of apoptosis, etc.

The doses of nucleic acid administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of nucleic acids of between 1 ng and 0.1 mg generally will be formulated and administered according to standard procedures. Other protocols for the administration of compositions will be known to one of ordinary skill in the art, in which the dose amount, schedule of injections, sites of injections, mode of administration (e.g., intra-tumoral) and the like vary from the foregoing. Administration of compositions to mammals other than humans, (e.g. for testing purposes or veterinary therapeutic purposes) is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a further preferred embodiment of the invention said composition further comprises at least one further therapeutic agent. Preferably said agent is a chemotherapeutic agent.

Preferably said agent is selected from the group consisting of: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

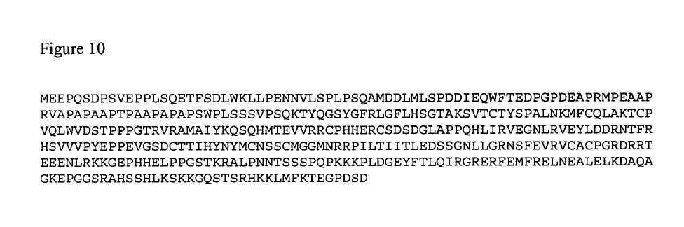

According to an aspect of the invention there is provided a screening method for the identification of an antagonist that inhibits the interaction of a p53 inhibitor polypeptide with a p53 polypeptide comprising the steps of:
i) forming a preparation comprising a polypeptide as represented by the amino acid sequence in FIG. 9, or a variant amino acid sequence, wherein said polypeptide comprises an amino acid sequence that includes the amino acid residue tyrosine 814 and a p53 polypeptide, or variant thereof, wherein said p53 polypeptide comprise amino acid residues 62-91 of the amino acid sequence represented in FIG. 10a;
ii) adding at least one candidate agent to be tested; and
iii) determining the effect, or not, of said antagonist on the interaction of said polypeptide fragment with the p53 polypeptide.

In a preferred method of the invention said p53 inhibitor polypeptide comprises a part of the amino acid sequence as represented in FIG. 9 wherein said part comprises amino acid residue tyrosine 814.

In a further preferred method of the invention said p53 polypeptide comprises a part of the amino acid sequence represented in FIG. 10a wherein said part comprises amino acid residues 62-91 of the amino acid sequence represented in FIG. 10a.

In a preferred method of the invention said p53 polypeptide comprise an arginine amino acid residue at position 72 of the amino acid sequence represented in FIG. 10a.

In a further preferred method of the invention said agent is a polypeptide.

In a preferred method of the invention said polypeptide is an antibody or active binding part thereof. Preferably said antibody or binding part is a monoclonal antibody.

Preferably, said antibody interferes with the binding of said p53 inhibitor polypeptide with p53 at tyrosine 814 and amino acid residues 62-91 of p53.

In a preferred method of the invention said antibody fragment is a single chain antibody variable region fragment or a domain antibody fragment.

It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a further preferred embodiment of the invention said antibody is a humanised or chimeric antibody.

A chimeric antibody is produced by recombinant methods to contain the variable region of an antibody with an invariant or constant region of a human antibody. A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred method of the invention said agent is a peptide, preferably a modified peptide.

It will be apparent to one skilled in the art that modification to the amino acid sequence of peptides which modulate the interaction of iASPP and p53 could enhance the binding and/or stability of the peptide with respect to its target sequence. In addition, modification of the peptide may also increase the in vivo stability of the peptide thereby reducing the effective amount of peptide necessary to induce apoptosis. This would advantageously reduce undesirable side effects which may result in vivo. Modifications include, by example and not by way of limitation, acetylation and amidation.

In a preferred method of the invention said peptide is acetylated. Preferably said acetylation is to the amino terminus of said peptide.

In a further preferred method of the invention said peptide is amidated. Preferably said amidation is to the carboxyl-terminus of said peptide.

In a further preferred method of the invention said peptide is modified by both acetylation and amidation.

Alternatively, or preferably, said modification includes the use of modified amino acids in the production of recombinant or synthetic forms of peptides. It will be apparent to one skilled in the art that modified amino acids include, by way of example and not by way of limitation, 4-hydroxyproline, 5-hydroxylysine, N⁶-acetyllysine, N⁶-methyllysine, N⁶, N6-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy. Alternatively, peptides could be modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred method of the invention peptides according to the invention are modified by cyclisation.

In a further preferred method of the invention said agent is an aptamer. Nucleic acids have both linear sequence structure and a three dimensional structure which in part is determined by the linear sequence and also the environment in which these molecules are located. Conventional therapeutic molecules are small molecules, for example, peptides, polypeptides, or antibodies, which bind target molecules to produce agonistic or antagonistic effects. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules, which comprise a binding domain for a target molecule. A screening method to identify aptamers is described in U.S. Pat. No. 5,270,163 which is incorporated by reference. Aptamers are typically oligonucleotides which may be single stranded oligodeoxynucleotides, oligoribonucleotides, or modified oligodeoxynucleotide or oligoribonucleotides.

The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The aptamers of the invention are synthesised using conventional phosphodiester linked nucleotides and synthesised using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. The binding of aptamers to a target polypeptide is readily tested by assays hereindisclosed.

In a preferred method of the invention said method further comprises a step wherein said agent is tested for activity with respect to a second different p53 polypeptide variant, preferably said p53 variant is modified by substitution of an amino acid residue encoded by codon 72 of the nucleic acid sequence as represented in FIG. 10b.

In a preferred method of the invention said p53 variant varies at codon 72 wherein said codon encodes an arginine or proline amino acid residue.

According to a further aspect of the invention there is provided a nucleic acid molecule as represented by the nucleic acid sequence in FIG. 8, or a nucleic acid molecule that hybridises under stringent hybridisation conditions to a nucleic acid molecule as represented in FIG. 8, wherein said nucleic acid encodes a peptide fragment which includes amino acid residue tyrosine 814 of the amino acid sequence shown in FIG. 8.

In a preferred embodiment of the invention said peptide fragment is at least 8 amino acid residues in length.

In a further preferred embodiment of the invention said peptide fragment is between about 9 amino acid residues and 18 amino acid residues in length.

In still further preferred embodiment of the invention said peptide fragment is between about 18 amino acid residues and 32 amino acid residues in length.

According to a further aspect of the invention there is provided a peptide fragment encoded by a nucleic acid according to the invention.

According to a still further aspect of the invention there is provided an immunogenic composition comprising a nucleic acid or peptide according to the invention. Preferably said composition further comprises an adjuvant or carrier.

An adjuvant is a substance or procedure which augments specific immune responses to antigens by modulating the activity of immune cells. Examples of adjuvants include, by example only, Freunds adjuvant, muramyl dipeptides, liposomes. The term carrier is construed in the following manner. A carrier is an immunogenic molecule which, when bound to a second molecule augments immune responses to the latter. Some antigens are not intrinsically immunogenic (i.e. not immunogenic in their own right) yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen. Helper T-cells can also stimulate other immune cells such as cytotoxic T-cells, and a carrier can fulfil an analogous role in generating cell-mediated immunity as well as antibodies.

According to a further aspect of the invention there is provided a method for preparing a hybridoma cell-line producing monoclonal antibodies comprising the steps of:
i) immunising an immunocompetent mammal with a nucleic acid, peptide or immunogenic composition according to the invention;
ii) fusing lymphocytes of the immunised immunocompetent mammal with myeloma cells to form hybridoma cells;
iii) screening monoclonal antibodies produced by the hybridoma cells of step (ii) for binding activity to the immunogen in (i);
iv) culturing the hybridoma cells to proliferate and/or to secrete said monoclonal antibody; and
v) recovering the monoclonal antibody from the culture supernatant.

Preferably, said immunocompetent mammal is a rodent, for example a mouse, rat or hamster.

According to a further aspect of the invention there is provided a hybridoma cell-line obtainable by the method according to the invention.

According to a still further aspect of the invention there is provided an antibody obtained from the hybridoma cell-line according to the invention.

According to a further aspect of the invention there is provided a method for the treatment of an animal which would benefit from a stimulation of apoptosis comprising administering a nucleic acid molecule or polypeptide or composition according to the invention

According to a further aspect of the invention there is provided a method for the immunisation of an animal comprising administering a nucleic acid or peptide or immunogenic composition according to the invention.

In a preferred method of the invention said animal is a human.

In a further preferred method of the invention said treatment or immunisation is the treatment of cancer or vaccination against, cancer. Preferably, said cancer is breast cancer.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures:

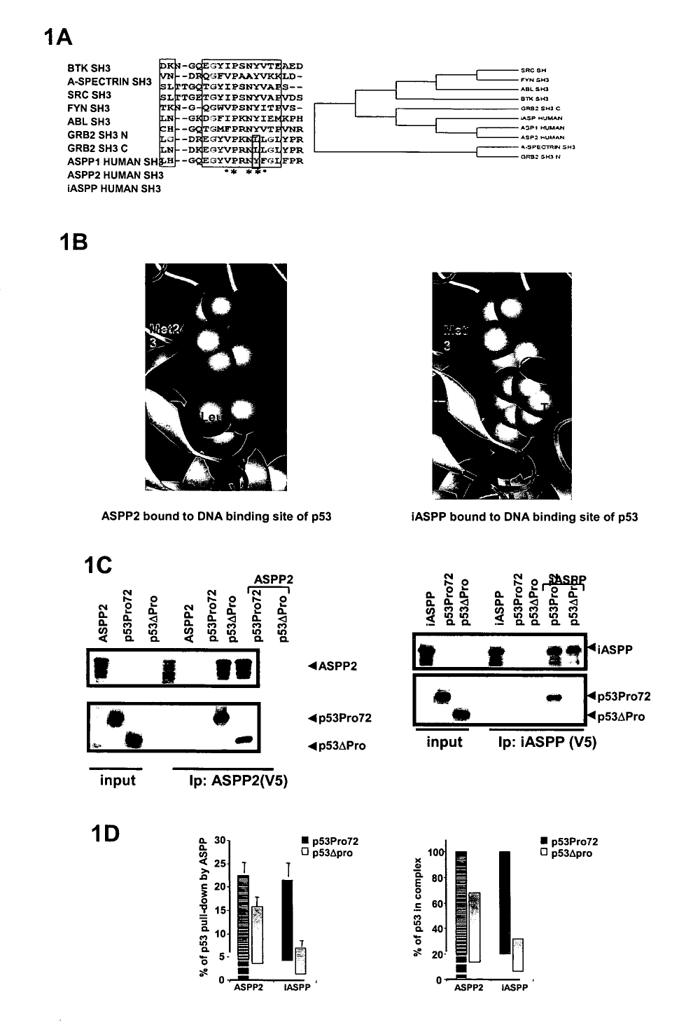

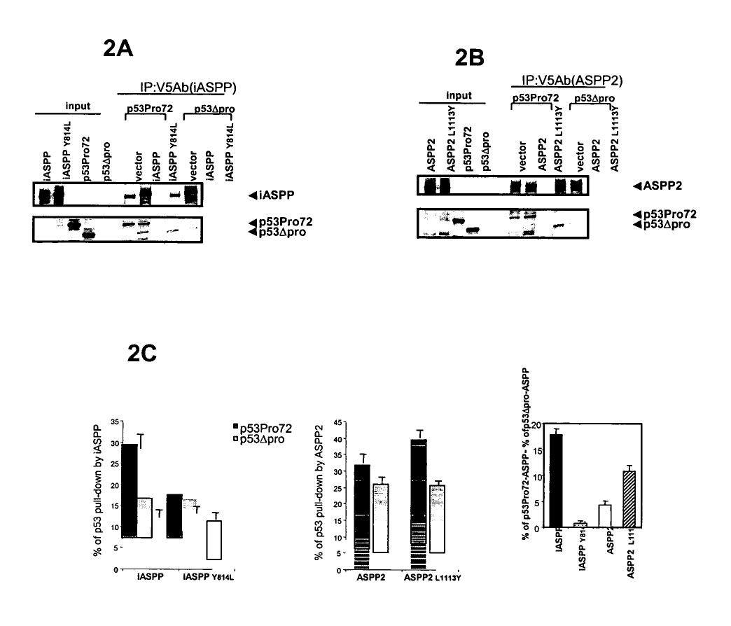

FIG. 1 illustrates that the SH3 domain of ASPP2 and iASPP interacts with the proline rich region of p53 with distinct binding affinity. A shows the alignment of the Y/L substitution in ASPP1 and ASPP2 with respect to iASPP and a selection of SH3 domains. The sequence homology of the SH3 domains among this group of proteins is illustrated in the right panel with a phylogenetic tree. The residues that contact proline rich region sequence is indicated as star and dot The dot indicates conserved hydrophobic residues near the contact site, while the star shows those residues that are critical to poly-proline interactions. The critical Y/L residue in the SH3 domain of the ASPP family of proteins is boxed. The computer modelling of the interactions between M243 of p53 and L1113 of ASPP2 and Y814 of iASPP is shown in B. Using in vitro translated, [³⁵S]methionine labelled ASPP2, iASPP, p53Pro72 and p53Δpro, the ability of ASPP2 and iASPP to bind p53Pro72 versus p53Δpro was measured (C). An aliquote of in vitro translated lysate is labelled as input. The signals derived from immunoprecipitates are labelled as IP. The expression levels of ASPP2, iASPP, p53Pro72 and p53Δpro are indicated with arrows. The signals from FIG. 1B were quantified using phosphor-imager (Molecular Imager FX, Biorad). The percentage of p53Pro72 versus p53Δpro in complex with ASPP2 or iASPP was calculated as that described in the experimental procedure (D, left panel). To compare the binding specificity of ASPP2 and iASPP to the proline rich region of p53, the amount of p53Pro72 in complex with ASPP2 or iASPP was used to set the value of 100% (D, right panel). The amount of p53Δpro in complex with ASPP2 or iASPP was obtained by using the signals obtained in p53Δpro panel to divide the ones from p53Pro72 (D, left panel).

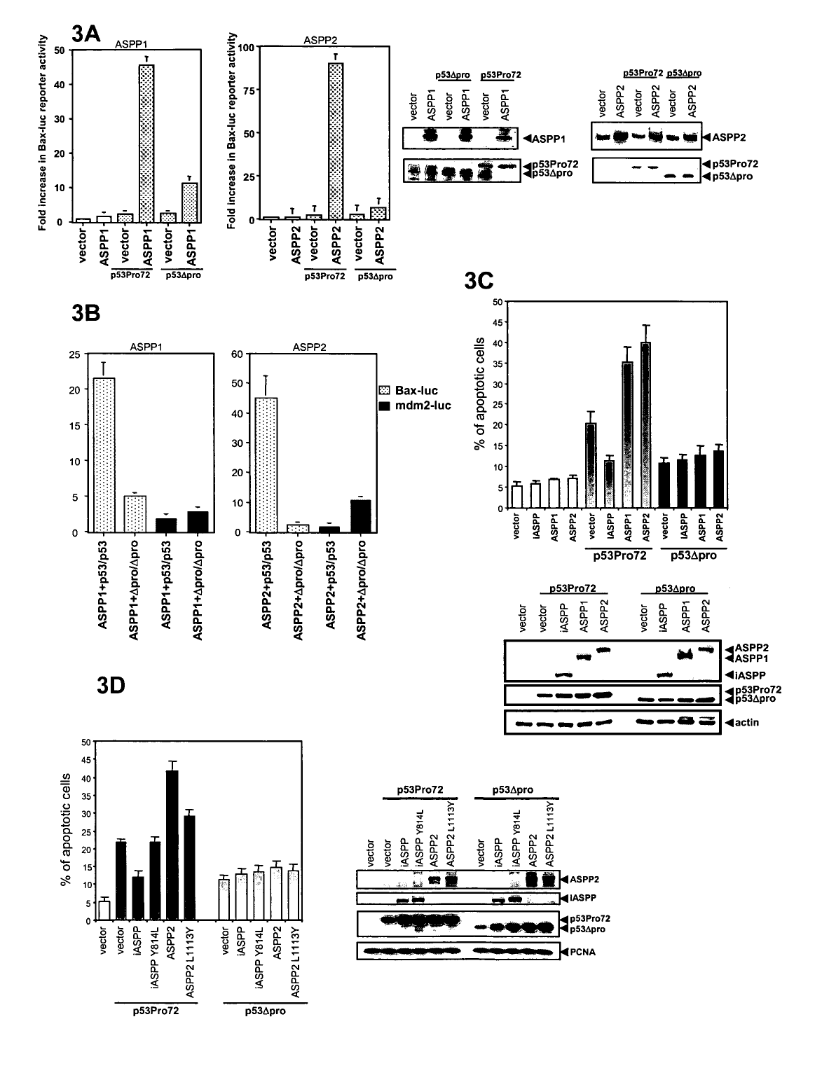

FIG. 2 illustrates that Y814 in the SH3 domain of iASPP regulates its binding specificity to the proline rich region of p53. In vitro immunoprecipitations shows that mutation of residue 814 of iASPP reduced its ability to complex with p53, whereas under the same conditions the efficiency of iASPP binding to p53Δpro was not affected by this mutation (A).

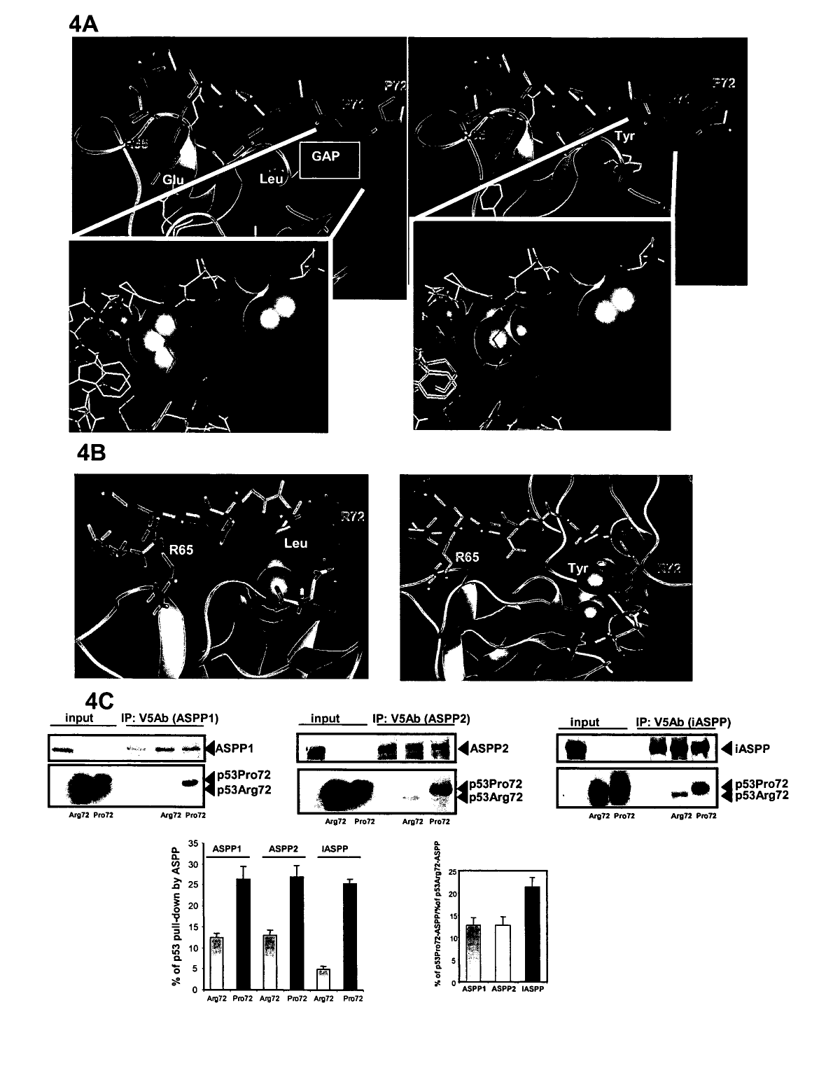

FIG. 3 illustrates that the proline rich region sequence of p53 is required for the ASPP family of proteins and their mutants to regulate the transactivation (A and B) and apoptotic (C and D) function of p53. In Saos-2 cells, the effects of the ASPP family of proteins and their mutants on the transactivation (A and B) and apoptotic function (C and D) of p53 versus pS3Δpro was compared. The expression plasmids used in the assays are indicated and their protein expression levels are shown in the immunoblots. The migrations of individual proteins are indicated by arrows.

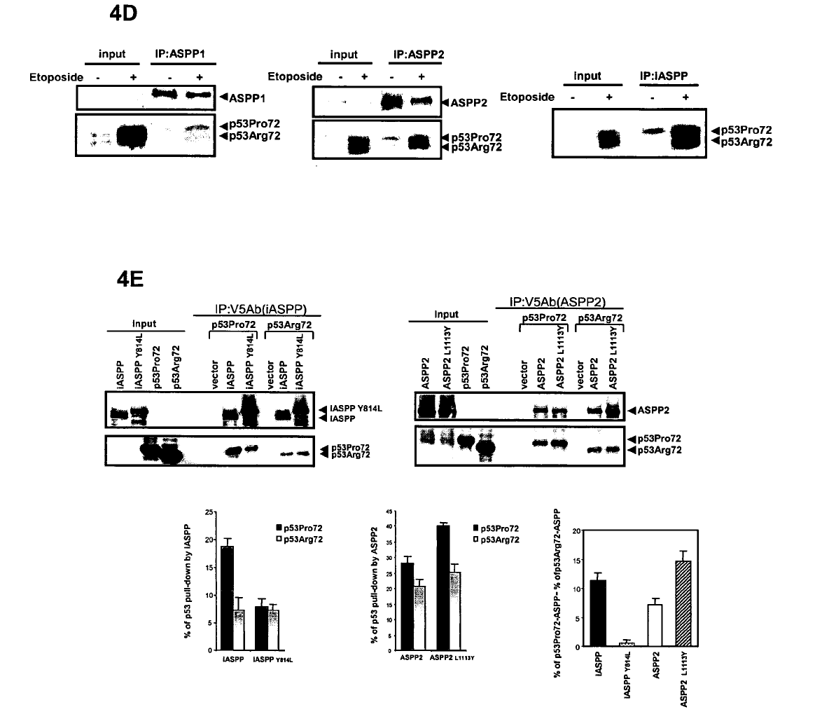

FIG. 4 illustrates computer modelling to show the importance of Y814 of iASPP and L1113 of ASPP2 in contacting Proline (A) or Arginine (B) of p53. The ASPP proteins have higher binding affinity to p53Pro72 than p53Arg72 in vitro (C) and in vivo (D). pS3Pro72, p53Arg72, ASPP1, ASPP2 and iASPP were in vitro translated and labelled with [³⁵S]methionine. ASPP1, ASPP2 and iASPP were all tagged with the V5 epitope and they were immunoprecipitated with antibody to V5 (IP: V5). The percentage of p53Pro72 or p53Arg72 in complex with ASPP1, ASPP2 and iASPP was calculated as that described in experimental procedure. The abilities of individual ASPP proteins to selectively complex with endogenous p53Pro72 were detected in a colorectal cell line RKO which expresses p53Pro72 and p53Arg72 at similar levels (D). RKO cells treated with etoposide (10 μM) for 8 hours are labelled as (+). The antibodies used to immunoprecipitate endogenous ASPP1, ASPP2 and iASPP were rabbit polyclonal antibodies 1.88, DX77 and iASPP.18, respectively and the amounts of ASPP1, ASPP2 and iASPP proteins immunoprecipitated by the antibodies were detected with mouse monoclonal antibodies LX54.2, DX54.10 and LX049 respectively. p53Pro72 and p53Arg72 were detected by the antibody DO.1. The ability of ASPP2/L1113Y and iASPP/Y814 to complex with p53Pro72 versus p53Arg72 was analysed in FIG. 4E. The percentage of ASPP2, iASPP and their mutants in complex with the two p53 polymorphic variants of p53 was calculated as that described in FIG. 1 and experimental procedures.

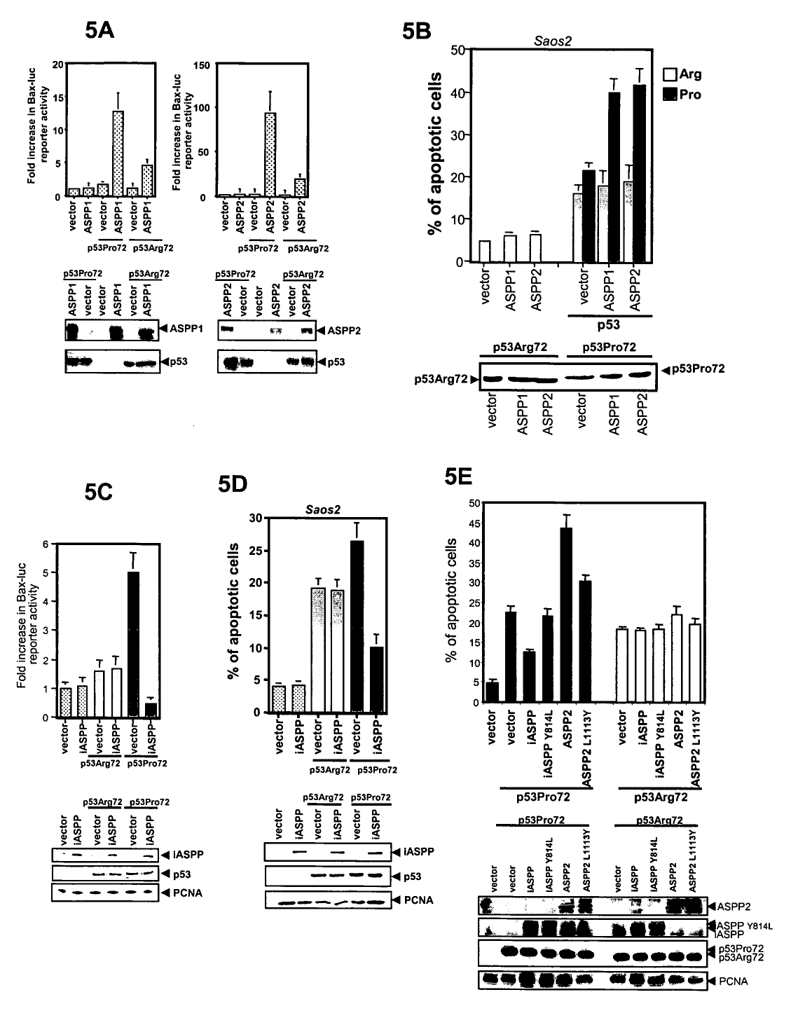

FIG. 5 illustrates that the ASPP family proteins selectively regulate the transactivation (A and C) and apoptotic functions (B, D and E) of p53Pro72. Saos-2 cells were transfected with p53Pro72 or p53Arg72, in the presence or absence of ASPP1, ASPP2, iASPP or their mutants as indicated. The bar graphs represent the mean value of at least three independent experiments. The expression levels of p53Pro72, p53Arg72, ASPP1, ASPP2, iASPP and their mutants are shown in the lower panels.

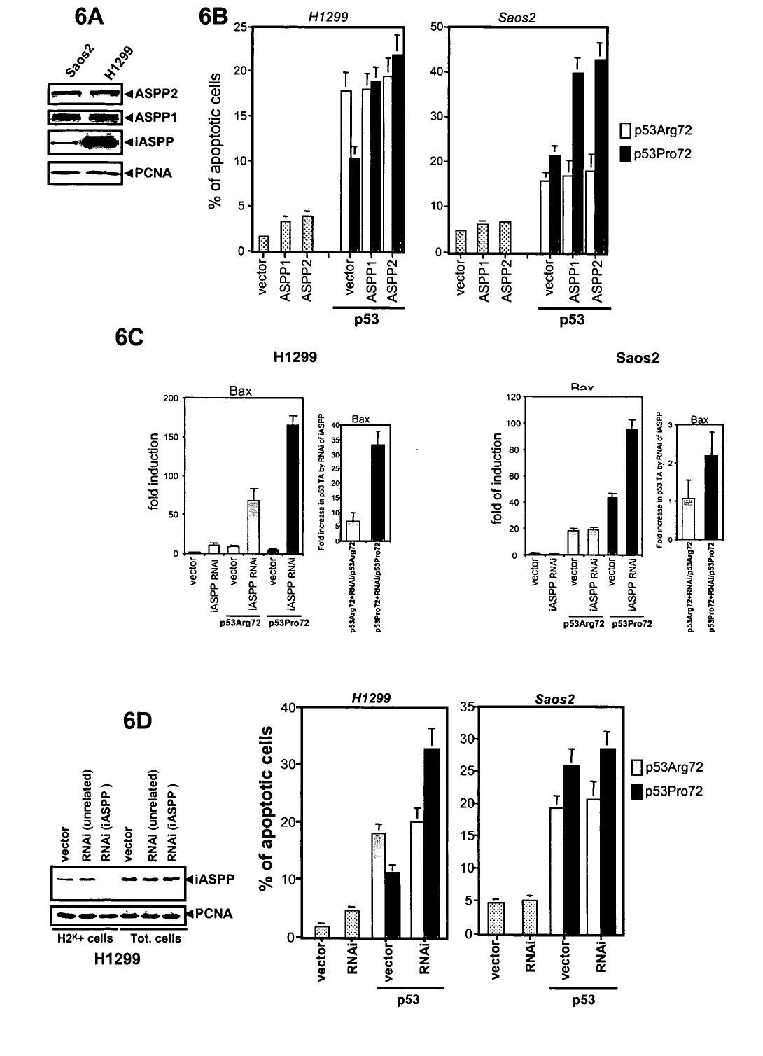

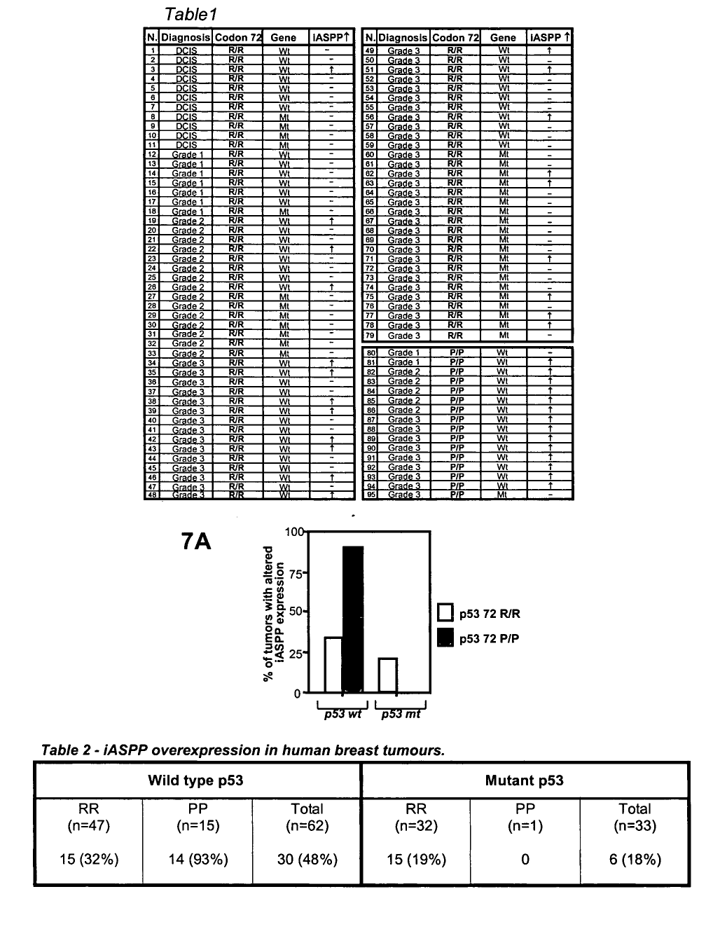

FIG. 6 illustrates that the endogenous iASPP expression level dictates the activities of p53Arg72 and p53Pro72. Western blot shows the expression levels of the ASPP family members in H1299 and Saos-2 cells (A). Expression of ASPP1 and ASPP2 enhanced the apoptotic function of p53Pro72 to a similar level as with p53Arg72 in H1299 cells (B). RNAi of iASPP enhanced the ability of p53Pro72 to transactivate Bax promoter in H1299 and Saos-2 cells more than with p53Arg72 (C). RNAi of iASPP also enhanced the apoptotic function of p53Pro72 but less so with p53Arg72 in H1299 cells. Under the same conditions, much smaller effects were observed with RNAi of iASPP in Saos-2 cells (D). The ability of RNAi of iASPP to reduce the expression of endogenous iASPP is shown in the left panel of FIG. 6D. H1299 cells were transfected with pSuper plasmid expressing RNAi of iASPP together with the cell surface marker H2K to allow the separation of transfected cells (H2K+ cells) from the un-transfected cells (H2K-cells).

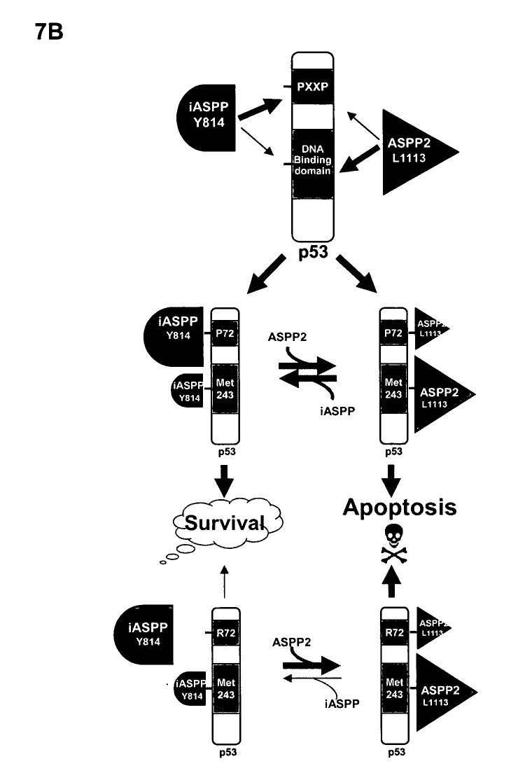

FIG. 7 illustrates (A) The bar graph shows the frequency of iASPP overexpressing in different category of tumour samples in comparison to their matched normal sample. The percentage was derived from table 2. (B) A diagram illustrates how ASPP2 and iASPP differentially regulate the apoptotic function of p53 codon 72 polymorphic variants;

FIG. 8 is the nucleic acid sequence of human iASPP;

FIG. 9 is the amino acid sequence of human iASPP;

FIG. 10 is the amino acid sequence of human p53; and

Table 1 and Table 2 illustrate mRNA expression of iASPP in human breast-tumor samples (DCIS, grade1-2-3) expressing either wild type or mutant p53. Up arrows represent overexpression of iASPP mRNA in comparison with their matched normal samples. Table 2 shows the percentage of tumour samples homozygous for p53Pro72 (PP) or p53Arg72 (RR) overexpressing iASPP with either wild type or mutant p53. “n” represents the number of samples in each category.

### Experimental Procedures

### Cells and Antibodies

Cells were grown in DMEM supplemented with 10% FCS. DO-1 is a mouse anti-p53 antibody. The V5 epitope is recognised by the mouse monoclonal antibody V5. CD20Leu is an FITC conjugated monoclonal antibody specific for the cell surface marker CD20 (Becton Dickinson). The mouse and rabbit antibodies to ASPP1 and ASPP2 were described previously (Rabbit anti-ASPP1 antibody pAb ASPP1.88, Rabbit anti-ASPP2 antibody pAb DX77 Rabbit anti-iASPP antibody pAb iASPP.18, mouse monoclonal anti-ASPP1 antibody LX011, mouse monoclonal anti-ASPP2 antibody DX54.10, mouse monoclonal anti-iASPP antibody miASPP49.3)^{2,28,29}.

### Plasmids

ASPP1, ASPP2, ASPP2L1113Y, iASPP and iASPPY814L expression plasmid were tagged with the V5 epitope. For transient expression of p53 the pCB6 plasmid was used, whereas the p53Arg72 and p53Pro72 used for the in vitro binding assays were cloned in pSP65 vector. p53Δpro was cloned in pcDNA3 vector.

### Transactivation Assays

Saos-2 or H1299 cells (5×10⁵) were plated 24 hours prior to transfection in 6 cm dishes. All transactivation assays contained 1 μg of reporter plasmid. 50 ng of p53Pro72 or p53Arg72, 4 μg of ASPP1 or ASPP2, 500 ng of iASPP expression plasmids were used as indicated. In FIG. 5C, cells were also co-transfected with 3 μg of pSuper plasmid containing iASPP RNAi as indicated. Cells were lysed in Reporter Lysis Buffer 16-24 hours after transfection and assayed using the Luciferase Assay kit (Promega, WI, USA). The fold activation of a particular reporter was determined by the activity of the transfected plasmid divided by the activity of vector alone.

### Flow Cytometry

Cells (10⁶) were plated 24-48 hours prior to transfection in 10 cm plates. All cells were transfected with 2 μg of CD20 expressing plasmid as a transfection marker. Transfection consisted of 1 μg of human p53Pro72 or p53Arg72, 2 μg of p53□pro, 10 μg of ASPP1, ASPP2 or ASPP2L1113Y, 1 μg of iASPP or iASPPY814L and 8 μg of pSuper plasmid containing RNAi of iASPP as indicated. 36 hours after the transfection, both attached and floating cells were harvested and analysed as previously described¹⁵.

### Protein Biochemistry

For western blotting, cells were lysed in either Nonidet P-40 (NP40) lysis buffer or luciferase reporter lysis buffer (Promega). Between 15-50 μg of protein extract was loaded on SDS-PAGE gels. For immunoprecipitation, cells were lysed in NP40 lysis buffer and pre-cleared with protein G beads for 1 hour at 4° C. The protein concentration was determined and then 1-2 mg of the extract was incubated with antibody pre-bound to protein G beads for 4 hours or overnight at 4° C. The beads were washed twice in NP40 lysis buffer and twice in NET buffer. The IP beads were mixed with 5× sample buffer and loaded onto an SDS-polyacrylamide gel. The gels were transferred (wet) to Protran nitrocellulose membranes, and the resulting blots were incubated first with primary antibody and subsequently with the appropriate secondary HRP conjugated antibody (Dako). The blot was exposed to hyperfilm following the use of ECL substrate solution (Amersham Life Science). The expression of endogenous ASPP1, ASPP2, iASPP, p53Pro72 and p53Arg72 were detected by using antibodies specific to these proteins derived from different species from that used in IP,

### In Vitro Translation and Immunoprecipitation

p53Pro72, p53Arg72, p53Δpro, ASPP1, ASPP2, ASPP2L1113Y, iASPP and iASPPY814L were in vitro translated and labeled with [³⁵S]methionine using the TNT T7 and TNT sp6 Quick coupled transcription-translation systems (Promega). The lysates containing indicated proteins were incubated at 30° C. for 1 h. The anti-V5 antibody immobilized on protein G-agarose beads was added to the binding reaction mixtures and incubated on a rotating wheel at 4° C. for 16 h. The beads were then washed with PBS. The bound proteins were released in SDS gel sample buffer and analyzed by SDS-10% polyacrylamide gel electrophoresis. Results were visualized by autoradiography.

Construction and Transfection of siRNA of iASPP

Oligonucleotides (19 bp) derived from iASPP were ligated into pSuper expression plasmids as described previously³⁰. The plasmids containing correct 19 bp oligonucleotides of iASPP were confirmed by sequencing. The sequences of iASPP sense and antisense oligonucleotides used in this study are as follow (lowercase indicates the vector sequence from pSuper; upper case indicates the target sequence for the RNAi):

Sense (S) and Antisense (A) oligos for iASPP

S:
5′gatccccTGTCAACTCCCCCGACAGCttcaagagaGCTGTCGGGGGAG
TTGACAtttttggaaa 3′
A:
5′agcttttccaaaaaTGTCAACTCCCCCGACAGCtctcttgaaGCTGTC
GGGGGAGTTGACAggg 3′

For transfection, 1×10⁶ H1299 or Saos2 cells were plated into 10 cm dishes. Cells were transfected with 2.5 μg of pMACS H-2K^{K} alongside either pSuper or pSuper-si-RNA iASPP (10 μg). 48 h after transfection, cells expressing the pMACS H-2K^{K} plasmid were separated using the MACS system (Miltenyi Biotec) according to the manufacturer's instructions. This gave rise to two populations of cells: H-2K^{K} expressing (transfected) cells and non-expressing (non-transfected cells). Both cell populations were lysed with RIPA buffer on ice for 30 minutes followed by centrifugation at 20 000 g for 30 minutes at 4° C.

### Real Time RT-PCR of Tumour and Matched Normal Controls

The breast cancers were all ductal carcinomas of no special type. The presence of an adequate proportion of tumour tissue was confirmed histologically prior to analysis. Codon 72 single nucleotide polymorphism was performed as described previously³¹. Mutations in p53 were analysed by single strand conformational polymorphism (SSCP) and sequencing as described. Expression of the ASPP family members was performed using TaqMan PCR. The primer sequences are as follows.

iASPP

| | |
|---|---|
| forward: | caggcggtgaaggagatgaacg |
| reverse: | aaatccacgatagagtagttggcgc |
| probe: | [FAM]-cccgagccagcccaacgagg-[TAMRA] |

### EXAMPLE 1

### ASPP2 and iASPP Have Distinct Binding Preference to the Proline-Rich Region and DNA Binding Domain of p53

Alignment of the SH3 domains of the ASPP family members with those SH3 from other proteins reveals the presence of Leu instead of the highly conserved Tyr at residues 1075 and 1113 for ASPP1 and ASPP2 respectively. Interestingly, the corresponding residue in iASPP is Tyr (Y814) (FIG. 1A). The Leu (1113) in ASPP2 can accommodate the side chain of Met (243) found in the DNA binding region of p53. In contrast, iASPP contains the classical SH3 domain sequence, where the Tyr may cause clashes with the p53-Met (FIG. 1B). Hence we tested whether the proline rich region of p53 could be the second binding site for the ASPP family members using in vitro translated p53Pro72, p53Δpro which contains an internal deletion of the proline-rich region of p53 (residues 62-91)⁷, ASPP2 and iASPP. The binding affinity of ASPP2 and iASPP to the proline rich region of p53 was also compared. In support of our theory, there was a large difference in the amount of p53Pro72 and p53Δpro in complex with ASPP2 and iASPP (FIG. 1C). To assess the requirement for the proline rich region of p53 in mediating the interaction of p53 with ASPP2 and iASPP, the percentage of p53Pro72/ASPP2 and p53Pro72/iASPP was arbitrarily set as 100%. As shown in FIG. 1D, Both ASPP2 and iASPP interacted with p53Pro72 and p53Δpro. However deletion of residues 62-91 (p53Δpro) reduced binding to iASPP to 26% of this. In contrast, the binding ability of p53Δpro to ASPP2 remained at around 70%. These results suggest that the ASPP family members bind to both the proline rich region and the DNA binding domain of p53. Furthermore, iASPP has higher binding affinity to the proline rich region of p53 while ASPP2 favours the DNA binding region of p53.

### EXAMPLE 2

### Y814 in the SH3 Domain of iASPP Determines its Binding Specificity to the Proline Rich Region of p53

To investigate whether the amino acid sequence difference found in the SH3 domain of ASPP2 (L1113) and iASPP (Y814) was one of the determining factors for the binding specificity of iASPP versus ASPP2 to the proline rich region of p53, we used oligonucleotide-directed mutagenesis to introduce an amino acid interchange between iASPP and ASPP2. Y814 of iASPP was changed to Leu while Leu 1113 of ASPP2 was changed to Tyr and the efficiency with which ASPP2 and iASPP bind to p53 and p53Δpro was compared to ASPP2/L1113Y and iASPP/Y814L in in vitro assays. Interestingly, the amount of p53 in complex with iASPP/Y814L was less than that in complex with iASPP, even though slightly more iASPP/Y814L was immunoprecipitated. Under the same conditions, similar amounts of p53Δpro were co-immunoprecipitated with iASPP and iASPP/Y814L (FIG. 2A). The efficiency of iASPP binding to p53□pro was not affected by this mutation (FIG. 2A, lower panel). In contrast, substitution of Tyr for Leu in the SH3 domain of ASPP2 at residue 1113 did not affect its binding to p53Pro72 (FIG. 2B). After normalisation for protein input, it is clear that the amount of p53Pro72 in complex with iASPP/Y814L is very similar to that of p53Δpro (FIG. 2C). An increase in p53 binding was seen with ASPP2/L1113Y compared to ASPP2 although the extent of increase varied among different experiments performed. The difference in the percentage of p53Pro72 versus p53□pro in complex with ASPP2 and ASPP2/L1113Y is 4% and 8% respectively (FIG. 2C). Together, these results suggest that Y814 of iASPP is critical for its ability to bind the proline rich region of p53. The modest increase in the ability of ASPP2/L1113Y to bind to p53Pro72 but not p53Δpro is consistent with the hypothesis that Tyr1113 of ASPP2 favours the interaction between its SH3 domain and the proline rich region of p53.

### EXAMPLE 3

### The Proline Rich Region of p53 is Required for the ASPP Family of Proteins to Regulate the Apoptotic Function of p53

The biological consequences of the interaction between the proline rich region of p53 and the ASPP family of proteins were tested using p53Pro72 and p53Δpro. Consistent with our previous observations, expression of ASPP1 or ASPP2 stimulated the transactivating activity of p53Pro72 on the human promoters of Bax but not mdm2. However, no stimulatory effects on p53Δpro were observed even though similar amounts of ASPP1 and ASPP2 were expressed (FIGS. 3A and 3B). Similarly, the apoptotic function of p53Δpro was not affected by the expression of the ASPP family of proteins even though the co-expression of ASPP1 and ASPP2 stimulated and iASPP inhibited the apoptotic function of p53Pro72 under the same conditions (FIG. 3C). These findings suggest that the proline rich region of p53 is required for the ASPP family of proteins to regulate the transactivating and apoptotic functions of p53.

To further demonstrate the importance of binding to the proline rich region of p53 in the iASPP-dependent inhibition of the apoptotic function of p53Pro72, we used flow cytometry to test the ability of iASPP/Y814L to inhibit p53-dependent apoptosis. Consistent with results from the binding assays, iASPP/Y814L has almost completely lost its ability to inhibit apoptosis induced by p53Pro72 (FIG. 3D). The failure to inhibit p53Pro72 induced apoptosis by iASPP/Y814L was not due to a lack of protein expression. Importantly, iASPP, ASPP2, iASPP/Y814L and ASPP2/L1113Y had no effects on the apoptotic function of p53Δpro (FIG. 3D). These results demonstrate that iASPP inhibits the apoptotic function of p53 predominantly through its ability to bind the proline rich region of p53 and Y814 of iASPP plays a pivotal role in controlling this activity of iASPP. Furthermore, binding to the DNA binding domain of p53 is not sufficient for ASPP1 and ASPP2 to enhance the apoptotic function of p53. The proline rich region of p53 is required for ASPP1 and ASPP2 to stimulate the transactivation and apoptotic functions of p53. The reduced pro-apoptotic function of ASPP2/L1113Y also suggests that there is an inverse correlation between the pro-apoptotic function of ASPP2 and its ability to bind the proline rich region of p53. This could be one of the mechanistic determinants modulating the pro-apoptotic and anti-apoptotic properties of the ASPP family of proteins.

### EXAMPLE 4

### The SH3 Domain of the ASPP Family Members, in Particular iASPP, Selectively Binds p53Pro72 In Vitro and In Vivo

The proline rich region of p53 spans residues 62-91⁷. Interestingly, the proline residue at position 72 is part of the PXXP motif present in p53, implying that Pro72 is one of the critical amino acids in p53 which contacts iASPP. A computer based three-dimensional structure modelling study was carried out based on the existing co-crystal structure of ASPP2/p53 (PDB code 1YCS). Initially iASPP was modelled based on ASPP2 and docked to p53 based on the complex structure 1YCS. After minimization, the iASPP-p53 complex was investigated to explore the differences between the binding interface of the DNA-binding region of p53 and ASPP2 versus iASPP. To investigate which part of the proline rich region of p53 would bind to the ASPP family; both sequential and structural studies were performed. A region was identified that contains both an arginine and the proline at codon 72. This poly-proline helix was modelled and docked into ASPP2 and iASPP based on the structure of Grb2-SOS complex (1AZE). The structural investigations showed that, as expected from the analysis of other SH3-poly pro complexes the Y814 of iASPP is an important contact residue to the prolines. When this is mutated to a Leu as in ASPP2, a large gap develops and the hydrophobic/aromatic contacts are lost (FIG. 4A). Furthermore, when the proline at codon 72 is changed into Arginine, it would have a large impact on the interaction between p53 and iASPP. The impact on p53/ASPP2 interaction would be less profound due to the smaller side chain of Leu at 1113 of ASPP2 (FIG. 4B). These results suggest that the ASPP family members, iASPP in particular, have different binding affinity to the common p53 polymorphic variants, p53Pro72 and p53Arg72.

To formally test this hypothesis, we used in vitro translated p53Arg72, p53Pro72, ASPP1, ASPP2 and iASPP. As shown in FIG. 4C, ASPP1 and ASPP2 associated with p53Pro72 with higher efficiency than with p53Arg72 but the most marked difference was seen between iASPP/p53Pro72 and iASPP/p53Arg72, with a clearly preferential binding of iASPP to p53Pro72. The preferential binding between p53Pro72 and the ASPP family members was further investigated in RKO cells, a colorectal cell line expressing wild type p53 and heterozygous for p53Pro72/p53Arg72. As shown in FIG. 4D, the amount of pS3Pro72 co-immunoprecipitated with iASPP was clearly and reproducibly greater than that of p53Arg72, even though similar amounts of p53Pro72 and p53Arg72 were expressed in RKO cells. Selective binding of ASPP2 to p53Pro72 was also seen. A similar pattern of selectivity towards p53Pro72 was also seen with ASPP1 but to a much lesser extent.

Since Y814 of iASPP could potentially contact the proline at residue 72 of p53, the ability of iASPP/Y814L and ASPP2/L11113Y to bind to p53Pro72 and p53Arg72 was also tested. Consistent with previous findings, there was a clear reduction in the binding ability of iASPP/Y814L to p53Pro72 but not to p53Arg72 (FIG. 4E). Interestingly, ASPP2/L1113Y bound p53Pro72 slightly better than ASPP2. Under identical conditions, both ASPP2 and ASPP2/L1113Y bind to p53Arg72 with similar efficiency. These results are consistent with the finding that the ASPP family of proteins bind to the proline rich region of p53 and that iASPP has higher binding affinity to the proline rich region of p53Pro72 than that of ASPP2. Our data also demonstrate that the ASPP family of proteins, particularly iASPP, selectively interact in vitro and in vivo with the common polymorphic variants of p53. Key individual residues in the SH3 domain of iASPP (Y814) and ASPP2 (L1113) have an important role in mediating the selective interaction between the ASPP family of proteins and the two polymorphic variants of p53, p53Pro72 and p53Arg72.

### EXAMPLE 5

### ASPP Family Members Selectively Regulate the Activity of p53Pro72

Having established that the ASPP family members, particularly iASPP, selectively interact with the two codon 72 p53 polymorphic variants, we next investigated whether the two polymorphic variants of p53 are subject to differential functional regulation by the ASPP family of proteins. In Saos-2 cells, the transactivating activity of p53Pro72 on the promoters of Bax and PIG3 is similar to or greater than p53Arg72 when expressed alone (FIG. 5A). Furthermore, the ability of ASPP1 and ASPP2 to enhance the transactivating activity of p53Pro72 is much greater than of p53Arg72. Similarly, the apoptotic function of p53Pro72 was also efficiently stimulated by co-expression of ASPP1 and ASPP2. Under the same conditions however, ASPP1 and ASPP2 have minimal effects on the apoptotic function of p53Arg72 (FIG. 5B). Moreover, the effects of iASPP on the transactivating and apoptotic function of p53Pro72 are also much greater than that on p53Arg72 (FIGS. 5C and 5D). Consistent with in vivo binding results, expression of iASPP/Y814 and ASPP2/L1113Y had no effect on the apoptosis function of p53Arg72, even though expression of iASPP/Y814L failed to inhibit apoptosis induced by p53Pro72 whereas expression of ASPP2/L1113Y had a reduced co-activating effect on p53Pro72 (FIG. 5E). Together, these results clearly illustrate that the ASPP family of proteins selectively regulate both the transactivating and apoptotic functions of the polymorphic p53 variants, p53Pro72 and p53Arg72.

### EXAMPLE 6

### The Level of iASPP Dictates the Activities of Polymorphic Variants of p53

The ability of endogenous ASPP family of proteins in regulating the activities of the two polymorphic p53 variants in vivo was first tested by examining the expression levels of ASPP1, ASPP2 and iASPP in H1299 and Saos-2 cells. Although the levels of ASPP1 and ASPP2 were similar in both cell lines, H1299 cells express 3-5 folds more iASPP than Saos-2 cells (FIG. 6A). Interestingly, p53Arg72 was more active than p53Pro72 to induce apoptosis in H1299 cells. In Saos-2 cells where the expression levels of iASPP is only ⅕ of that seen in H1299 cells, p53Arg72 is not more active than p53Pro72 in induction of apoptosis (FIG. 6B). Consistent with this, exogenous expression of ASPP1 and ASPP2 enhanced the activity of pS3Pro72 to a level similar to that observed with p53Arg72 in H1299 cells (FIG. 6B, right panel), suggesting that the inhibitory activities of endogenous iASPP can be counteracted by over-expression of ASPP1 or ASPP2. These results clearly illustrate that the apoptotic function of the two p53 polymorphic variants is influenced by cell context and imply that the expression levels of iASPP in the cells influence the apoptotic function of the polymorphic p53 variants.

The more efficient binding of iASPP to pS3Pro72 than to p53Arg72, implies that p53Arg72 is less sensitive to the inhibitory effects of iASPP, a mechanism which potentially explains the relatively greater apoptosis-inducing activity of p53Arg72 in H1299 cells. The ability of iASPP to influence the activities of p53Pro72 and p53Arg72 was further tested using RNA interference to reduce the expression of endogenous iASPP. In Saos-2 cells, iASPP RNAi stimulated the transactivating activity of p53Arg72 and pS3Pro72 on the Bax promoter by 1 and 2 fold respectively (FIG. 6C). In H1299 cells, RNAi of iASPP enhanced the transactivation function of p53Arg72 and pS3Pro72 on Bax promoter by 7 and 33 fold. Similarly, RNAi of iASPP also had greater effects on the apoptotic function of p53 in H1299 cells than in Saos-2 cells. Reduced expression of endogenous iASPP by RNAi dramatically enhanced the apoptotic function of p53Pro72 in H1299 cells (FIG. 6D). In agreement with the finding that the expression level of iASPP is higher in H1299 cells than in Saos-2 cells, the extent of increase in apoptotic function of p53Pro72 induced by iASPP RNAi in Saos-2 cells is much smaller than in H1299 cells. These results demonstrate that the ASPP family members, iASPP in particular, are critical determinants of the apoptotic function of the two polymorphic variants of p53.

### EXAMPLE 7

### Over-Expression of iASPP is Significantly More Frequent in Tumours Homozygous for p53Pro72

To further investigate the biological and potentially clinical importance of our findings, we examined the expression levels of iASPP in a panel of matched normal and human breast carcinomas homozygous for p53Arg72 (n=62) or p53Pro72 (n=16) (table 1) using TaqMan (real-time) RT-PCR. Detailed analysis revealed that the frequency of over-expression of iASPP is significantly higher in cases with wild type p53 which are homozygous for p53Pro72 than in those homozygous for p53Arg72 in the germ-line (table 2). Whereas iASPP was over expressed in 90% of the tumours homozygous for p53Pro72 (14/15), only 32% (15/47) of the tumours homozygous for p53Arg72 over-expressed iASPP mRNA (table 1, table 2 and FIG. 7A). The difference in the frequency of iASPP over-expression between tumours homozygous for p53Pro72 and p53Arg72 is statistically significant (p<0.01). These results suggested that over-expression of iASPP could be one the mechanisms by which the tumour suppression function of p53Pro72 was inactivated in these tumours. Over-expression of iASPP may confer a selective growth advantage during oncogenesis of tumours homozygous for p53Pro72. AS such, molecules which can specifically disrupt the interaction between p53Pro72 and iASPP may allow reactivation of the apoptotic function p53 in these tumours.

Here we show that the proline rich region is the second binding site of the ASPP family members to p53. This initial observation led us to delineate a novel mechanism by which the ASPP family members regulate the apoptotic function of p53. We provide evidence that the proline rich region of p53 binds to iASPP more efficiently than to ASPP1 (data not shown) and ASPP2. This finding provides molecular insights into the mechanistic basis by which iASPP inhibit, while ASPP1 and ASPP2 stimulate, the apoptotic function of p53. The failure of iASPP/Y814L to bind the proline rich region of p53 and to inhibit the apoptotic function of p53 demonstrates for the first time that iASPP predominantly inhibits the apoptotic function of p53 through its ability to selectively bind the proline rich region of p53. In contrast previous studies showed that many proteins can stimulate the activities of p53 through their ability to interact with the proline rich region of p53. Binding to the proline rich region and enhance the acetylation of p53 is one of the mechanism by which p300 stimulates the transactivation function of p53¹⁸.The binding of corepressor mSin3a protein to the proline rich region of p53 can also increased the stability and transrepression function of p53¹⁹, one of the property of p53 which is closely linked to its apoptotic function²⁰. Additionally IKbΔ can bind to the proline rich region of p53 and enhance p53 mediated apoptosis. This interaction of IkbΔ requires the phosphorylation of p53 at Ser46²¹. We do not yet know the precise mechanism by which the proline rich region of p53 is involved in activating the full apoptotic function of p53. However the results shown in this study suggested that binding to the proline rich region of p53 and preventing other activators such as p300, Sin3A and IKbΔ from binding to the same region of p53 is perhaps one of the mechanisms by which iASPP inhibits the apoptotic function of p53.

In the absence of any co-crystal structure information between the proline rich region of p53 and the ASPP family members, it remains unclear whether the binding of iASPP to the proline rich region of p53 could change the protein conformation of p53 and alter the ability of p53 to bind DNA, ASPP1, ASPP2 and other interacting partners of p53. Nonetheless, the findings shown here provide an attractive possibility that binding to iASPP and abrogating its anti-apoptotic function is one of the reasons why the proline rich region sequence of p53 is required for its full apoptotic function. Future structural studies of p53 containing the proline rich region sequence are needed to provide a detailed molecular explanation of these questions. The failure of ASPP1 and ASPP2 to stimulate the apoptotic function of the proline rich region deleted p53 mutant, p53Δpro, as well as p53Arg72 suggested that counteracting the inhibitory activity of iASPP is one of the main mechanisms by which ASPP1 and ASPP2 stimulate the apoptotic function of p53 (FIG. 7B). The identification of different binding affinities among the ASPP family members to the two p53 binding sites, i.e. the proline rich region and the DNA binding domain, provide us an opportunity to look for molecules which can specifically disrupt the interaction between iASPP/p53 but not ASPP1/p53 or ASPP2/p53.

The findings reported here also reveal a novel insight into the mechanism by which the apoptosis function of the p53 polymorphic variants, p53Pro72 and p53Arg72, is regulated. The regulation of p53 by ASPP family proteins is evolutionarily conserved and the most conserved member of the ASPP family and the only ASPP family member present in C.elegans³ is iASPP. Hence it is interesting to note that iASPP has a higher binding affinity to p53Pro72 than to p53Arg72. The polymorphism of p53 at codon 72 only exists in human and p53Arg72 is human specific. Moreover, the frequency of the allele encoding p53Pro72 varies among different ethnic populations. The number of individuals homozygous for p53Pro72 is closely linked to latitude and is much higher in the black populations living near the equator, suggesting that p53Pro72 is selected in an environment with high levels of UV light^{22,23}. A molecular explanation of this selection may be that the ASPP family of proteins, iASPP in particular, selectively regulate the apoptotic function of p53Pro72. In response to various stress signals, two different pathways are involved in regulating the apoptotic function of p53Pro72 or p53Arg72. It was previously shown that the p53Arg72 preferentially localizes to the mitochondria²⁴. Here, we have identified an alternative pathway by which p53Arg72 is more active than p53Pro72 in induction of apoptosis and has, therefore, evolved in humans. The insensitivity of p53Arg72 to inhibition by iASPP implies that the most efficient way to inactivate the apoptotic function of p53Arg72 in human tumourigenesis is by intragenic mutation. Consistent with this hypothesis and previous publication, the percentage of tumours expressing mutant p53 that are homozygous for p53Arg72 is much higher than those with p53Pro72 (42% and 6% respectively) in the panel of breast tumours examined²⁵. Being a more potent inhibitor of p73, there is also a selective advantage to mutate p53Arg72 in tumours²⁶. In contrast, inactivation of p53Pro72 can occur by reduction in expression of ASPP1, ASPP2 or over-expression of iASPP, in addition to mutation in p53 itself. Therefore the ASPP family of proteins provided another level of regulation of p53Pro72. As a result, p53Pro72 is less prone to mutation than p53Arg72 in normal cells in response to signals that induce the apoptotic function of p53. This may be why the percentage of p53Pro72 homozygous carriers is highest in the ethnic populations that have evolved in an environment consistently exposed to high dose of p53 inducing agents such as UV radiation. Nevertheless, homozygosity for p53pro72 does not necessarily mean protection against p53 mutation in other types of cancer because the expression levels of the ASPP family proteins vary dramatically among different tissues (data not shown)²⁷. Only when the expression levels of the ASPP family members are taken into consideration, can clear conclusions be drawn on association of expression of polymorphic p53 variants with cancer susceptibility. The results shown here suggest the possibility of improved strategies to treat cancer according to their p53 polymorphism and ASPP expression patterns.

### REFERENCES

1. Slee, E. A., O'Connor, D. J. & Lu, X. To die or not to die: how does p53 decide? Oncogene 23, 2809-18 (2004).
2. Samuels-Lev, Y. et al. ASPP proteins specifically stimulate the apoptotic function of p53. Mol Cell 8, 781-94 (2001).
3. Bergamaschi, D. et al. iASPP oncoprotein is a key inhibitor of p53 conserved from worm to human. Nat Genet 33, 162-7 (2003).
4. Liu, Z. J., Zhang, Y., Zhang, X. B. & Yang, X. Abnormal mRNA expression of ASPP members in leukemia cell lines. Leukemia 18, 880 (2004).
5. Zhang, X., Wang, M., Zhou, C., Chen, S. & Wang, J. The expression of iASPP in acute leukemias. Leuk Res 29, 179-83 (2005).
6. Lossos, I. S., Natkunam, Y., Levy, R. & Lopez, C. D. Apoptosis stimulating protein of p53 (ASPP2) expression differs in diffuse large B-cell and follicular center lymphoma: correlation with clinical outcome. Leuk Lymphoma 43, 2309-17 (2002).
7. Walker, K. K. & Levine, A. J. Identification of the novel p53 functional domain which is necessary for efficient growth suppression. Proc. Natl. Acad. Sci. USA 93, 15335-15340 (1996).
8. Zhu, J., Jiang, J., Zhou, W., Zhu, K. & Chen, X. Differential regulation of cellular target genes by p53 devoid of the PXXP motifs with impaired apoptotic activity. Oncogene 18, 2149-2155 (1999).
9. Baptiste, N., Friedlander, P., Chen, X. & Prives, C. The proline-rich domain of p53 is required for cooperation with anti-neolastic agents to promote apoptosis of tumour cells. Oncogene 21, 9-21 (2002).
10. Venot, C. et al. The requirement for the p53 proline-rich functional domain for mediation of apoptosis is correlated with specific PIG3 gene transactivation and with transcriptional repression. EMBO 17, 4668-4679 (1998).
11. D'Erchia A, M., Pesole, G., Tullo, A., Saccone, C. & Sbisa, E. Guinea pig p53 mRNA: identification of new elements in coding and untranslated regions and their functional and evolutionary implications. Genomics 58, 50-64 (1999).
12. Noma, C., Miyoshi, Y., Taguchi, T., Tamaki, Y. & Noguchi, S. Association of p53 genetic polymorphism (Arg72Pro) with estrogen receptor positive breast cancer risk in Japanese women. Cancer Lett 210, 197-203 (2004).
13. Granja, F. et al. Proline homozygosity in codon 72 of p53 is a factor of susceptibility for thyroid cancer. Cancer Lett 210, 151-7 (2004).
14. Agorastos, T. et al. P53 codon 72 polymorphism and correlation with ovarian and endometrial cancer in Greek women. Eur J Cancer Prev 13, 277-80 (2004).
15. de Oliveira, W. R. et al. Association of p53 arginine polymorphism with skin cancer. Int J Dermatol 43, 489-93 (2004).
16. Matakidou, A., Eisen, T. & Houlston, R. S. TP53 polymorphisms and lung cancer risk: a systematic review and meta-analysis. Mutagenesis 18, 377-85 (2003).
17. Cenci, M. et al. p53 polymorphism at codon 72 is not a risk factor for cervical carcinogenesis in central Italy. Anticancer Res 23, 1385-7 (2003).
18. Chanet, R., Magana-Schwencke, N. & Fabre, F. Potential DNA-binding domains in the RAD18 gene product of Saccharomyces cerevisiae. Gene 74, 543-547 (1988).
19. Zilfou, J. T., Hoffman, W. H., Sank, M., George, D. L. & Murphy, M. The corepressor mSin3a interacts with the proline-rich domain of p53 and protects p53 from proteasome-mediated degradation. Mol Cell Biol 21, 3974-85 (2001).
20. Hsieh, J.-K. et al. Rb regulates the stability and the apoptotic function of p53 via mdm2. Mol. Cell 3, 181-193 (1999).
21. Chang, N. S. The non-ankyrin C terminus of Ikappa Balpha physically interacts with p53 in vivo and dissociates in response to apoptotic stress, hypoxia, DNA damage, and transforming growth factor-beta 1-mediated growth suppression. J Biol Chem 277, 10323-31 (2002).
22. Sjalander, A., Birgander, R., Kivela, A. & Beckman, G. p53 polymorphisms and haplotypes in different ethnic groups. Hum Hered 45, 144-9 (1995).
23. Beckman, G. et al. Is p53 polymorphism maintained by natural selection? Hum Hered 44, 266-70 (1994).
24. Dumont, P., Leu, J. I., Della Pietra, A. C., 3rd, George, D. L. & Murphy, M. The codon 72 polymorphic variants of p53 have markedly different apoptotic potential. Nat Genet 33, 357-65 (2003).
25. Langerod, A. et al. The TP53 codon 72 polymorphism may affect the function of TP53 mutations in breast carcinomas but not in colorectal carcinomas. Cancer Epidemiol Biomarkers Prev 11, 1684-8 (2002).
26. Marin, M. C. et al. A common polymorphism acts as an intragenic modifier of mutant p53 behaviour. Nat Genet 25, 47-54 (2000).
27. Iwabuchi, K., Li, B., Bartel, P. & Fields, S. Use of the two-hybrid system to identify the domain of p53 involved in oligomerization. Oncogene 8, 1693-1696 (1993).
28. Slee, E. A. et al. The N-terminus of a novel isoform of human iASPP is required for its cytoplasmic localization. Oncogene (2004).
29. Bergamaschi, D. et al. ASPP1 and ASPP2: common activators of p53 family members. Mol Cell Biol 24, 1341-50 (2004).
30. Brummelkamp, T. R., Bernards, R. & Agami, R. A system for stable expression of short interfering RNAs in mammalian cells. Science 296, 550-3 (2002).
31. Bergamaschi, D. et al. p53 polymorphism influences response in cancer chemotherapy via modulation of p73-dependent apoptosis. Cancer Cell 3, 387-402 (2003).

1. An isolated nucleic acid molecule comprising the nucleic acid sequence in FIG. 8 (SEQ ID NO: 17), or a nucleic acid molecule that hybridises under stringent hybridisation conditions to a polynucleotide complimentary to the sequence in FIG. 8 (SEQ ID NO: 17), wherein said nucleic acid is modified at a nucleotide codon that encodes for a tyrosine amino acid residue corresponding to position 814 of an iASPP amino acid sequence that comprises the amino acid sequence represented in FIG. 9 (SEQ ID NO: 16), such that the tyrosine corresponding to position 814 (SEQ ID NO: 16, position 337) is deleted or substituted by another amino acid.

2. A nucleic acid molecule according to claim 1, wherein said nucleic acid molecule comprises the nucleic acid sequence represented in FIG. 8 (SEQ ID NO: 17) that encodes the amino acid sequence in FIG. 9 (SEQ ID NO: 16), with the proviso that the codon for said tyrosine has been deleted or replaced with a codon for another amino acid.

3. A nucleic acid molecule according to claim 1, wherein said nucleic acid molecule consists of the nucleic acid sequence as represented in FIG. 8 (SEQ ID NO: 17) that encodes the amino acid sequence in FIG. 9 (SEQ ID NO: 16), with the proviso that the codon for said tyrosine has been deleted or replaced with a codon for another amino acid.

4. An expression vector comprising a nucleic acid molecule according to claim 1.

5. An isolated polypeptide comprising an amino acid sequence that is at least 95% identical to the amino acid sequence in FIG. 9 (SEQ ID NO: 16), with the proviso that the amino acid sequence of the polypeptide is modified by the deletion or substitution of at least amino acid residue tyrosine 814 (SEQ ID NO: 16, position 337) of the amino acid sequence represented in FIG. 9 (SEQ ID NO: 16).

6. A polypeptide according to claim 5, wherein said tyrosine amino acid residue is substituted with a leucine amino acid residue.

7. A variant polypeptide according to claim 5, wherein said variant polypeptide is modified, relative to the sequence in FIG. 9, only at amino acid residue tyrosine 814 (SEQ ID NO: 16, position 337).

8. (canceled)

9. (canceled)

10. A composition comprising a nucleic acid according to claim 1 and a pharmaceutically acceptable carrier.

11. A composition according to claim 10, wherein said composition further comprises at least one further therapeutic agent.

12. A composition according to claim 11, wherein said agent is a chemotherapeutic agent.

13. A composition according to claim 12, wherein said agent is selected from the group consisting of: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

14. A screening method for the identification of an antagonist that inhibits the interaction of a p53 inhibitor polypeptide with a p53 polypeptide comprising the steps of:
i) forming a preparation comprising a polypeptide as represented by the amino acid sequence in FIG. 9 (SEQ ID NO: 16), or a variant amino acid sequence, wherein said polypeptide comprises an amino acid sequence that includes the amino acid residue tyrosine 814 and a p53 polypeptide, or variant thereof, wherein said p53 polypeptide comprise amino acid residues 62-91 of the amino acid sequence represented in FIG. 10 (SEQ ID NO: 18);
ii) adding at least one candidate agent to be tested; and
iii) determining the effect, or not, of said antagonist on the interaction of said polypeptide fragment with the p53 polypeptide.

15. A method according to claim 14, wherein said p53 inhibitor polypeptide comprises a part of the amino acid sequence as represented in FIG. 9 (SEQ ID NO: 16) wherein said part comprises amino acid residue tyrosine 814.

16. A method according to claim 14, wherein said p53 polypeptide comprises a part of the amino acid sequence represented in FIG. 10 (SEQ ID NO: 18) wherein said part comprises amino acid residues 62-91 of the amino acid sequence represented in FIG. 10 (SEQ ID NO: 18).

17. A method according to claim 14, wherein said p53 polypeptide comprise an arginine or proline amino acid residue at position 72 of the amino acid sequence represented in FIG. 10 (SEQ ID NO: 18).

18. A method according to claim 14, wherein said agent is a polypeptide.

19. A method according to claim 18, wherein said polypeptide is an antibody or active binding part thereof.

20. A method according to claim 19, wherein said antibody or active binding part is a monoclonal antibody.

21. An method according to claim 19, wherein said antibody interferes with the binding of said p53 inhibitor polypeptide with p53 at tyrosine 814 and amino acid residues 62-91 of p53.

22. A method according to claim 19, wherein said antibody fragment is a single chain antibody variable region fragment or a domain antibody fragment.

23. A method according to claim 19, wherein said antibody is a humanised or chimeric antibody.

24. A method according to claim 14, wherein said agent is a peptide,

25. A method according to claim 24, wherein said peptide is a modified peptide.

26. A method according to claim 14, wherein said agent is an aptamer.

27. A method according to claim 14, wherein said method further comprises a step wherein said agent is tested for activity with respect to a second different p53 polypeptide variant,

28. A method according to claim 27, wherein said p53 variant is modified by substitution of an amino acid residue at position 72 of the amino acid sequence as represented in FIG. 10 (SEQ ID NO: 18).

29. A method according to claim 28, wherein said p53 variant vanes at codon 72 wherein said codon encodes an arginine or proline amino acid residue.

30. An isolated nucleic acid molecule comprising a coding portion of the nucleic acid sequence in FIG. 8 (SEQ ID NO: 17), or a nucleic acid molecule that hybridises under stringent hybridisation conditions to a polynucleotide complimentary to the sequence in FIG. 8 (SEQ ID NO: 17), wherein said nucleic acid encodes a peptide fragment at least 8 amino acid residues in length which includes amino acid residue tyrosine 814 (SEQ ID NO: 16, position 337) of the amino acid sequence shown in FIG. 9 (SEQ ID NO: 16).

31. (canceled)

32. A nucleic acid molecule according to claim 30, wherein said peptide fragment is between about 9 amino acid residues and about 30 amino acid residues in length.

33. A nucleic acid molecule according to claim 30, wherein said peptide fragment is between about 10 amino acid residues and about 19 amino acid residues in length.

34. A peptide of at least 8 amino acid residues that comprises an amino acid sequence selected from the group consisting of:
(a) peptide fragments of the amino acid sequence of FIG. 9 (SEQ ID NO: 16) that include tyrosine 814 (SEQ ID NO: 16, position 337); and
(b) variants of (a) that include said tyrosine and are encoded by a nucleic acid molecule that hybridizes under stringent hybridization conditions to a polynucleotide complementary to the nucleotide sequence in FIG. 8 (SEQ ID NO: 17)

35. An immunogenic composition comprising a peptide according to claim 34.

36. A composition according to claim 35, wherein said composition further comprises an adjuvant or carrier.

37. A method for preparing a hybridoma cell-line that produces monoclonal antibodies comprising the steps of:
i) immunising an immunocompetent mammal with a composition according to claim 36;
ii) fusing lymphocytes of the immunised immunocompetent mammal with myeloma cells to form hybridoma cells;
iii) screening monoclonal antibodies produced by the hybridoma cells of step (ii) for binding activity to the immunogen in (i).

38. A hybridoma cell-line obtainable by the method according to claim 37.

39. An antibody obtained from the hybridoma cell-line according to claim 38.

40. A method for the treatment of an animal which would benefit from a stimulation of apoptosis comprising administering to the animal a nucleic acid molecule according to claim 1.

41. A method for the immunisation of an animal comprising administering to the animal a composition according to claim 36.

42. A method according to claim 40, wherein said animal is a human.

43. A method according to claim 40, wherein said treatment is the treatment of cancer or vaccination against, cancer.

44. A method according to claim 43, wherein said cancer is breast cancer.

45. An antibody that specifically immunoreacts with a peptide fragment of at least 8 amino acid residues of the amino acid sequence of FIG. 9 (SEQ ID NO: 16) that include tyrosine 814 (SEQ ID NO: 16, position 337).

46. The antibody of claim 45 that is a monoclonal antibody.

47. The peptide of claim 34 that is from 9 to 30 amino acids in length.

48. A method for the treatment of an animal which would benefit from a stimulation of apoptosis comprising administering to the animal a composition that comprises a peptide according to claim 34 and a pharmaceutically acceptable carrier.

49. A composition comprising the polypeptide according to claim 5 and a pharmaceutically acceptable carrier.

50. A composition comprising a nucleic acid according to claim 30 and a pharmaceutically acceptable carrier.

51. A composition comprising a peptide according to claim 34 and a pharmaceutically acceptable carrier.
